(19) United States
(12) Patent Application Publication
(10) Pub. No.: US 2025/0302570 A1
(43) Pub. Date: Oct. 2, 2025

(54) REPROCESSING TRAY, FIXATION ELEMENT, AND REPROCESSING MACHINE

(71) Applicant: OLYMPUS Winter & Ibe GmbH, Hamburg (DE)

(72) Inventor: Annika HOLZSCHUH, Hamburg (DE)

(73) Assignee: OLYMPUS Winter & Ibe GmbH, Hamburg (DE)

(21) Appl. No.: 19/088,215

(22) Filed: Mar. 24, 2025

(30) Foreign Application Priority Data
Mar. 29, 2024 (DE) 10 2024 109 128.2

(51) Int. Cl. A61B 50/33; A61B 50/20; A61B 90/00

(52) U.S. Cl. CPC A61B 50/33; A61B 50/20; A61B 90/08; A61B 2090/0807

(57) ABSTRACT

A reprocessing tray for holding a medical instrument. The reprocessing tray including a tray body; and a plurality of fixation elements fixed to a surface of the tray body. Wherein each of the plurality of fixation elements include a visual indicia. The visual indicia indicating one or more of the following: a sequence, in which the medical instrument is to be brought into contact with each of the plurality of fixation elements, and a portion of the medical instrument, which is to be brought into contact with each of the plurality of fixation elements.

### CROSS-REFERENCE TO RELATED APPLICATION

The present application is based upon and claims the benefit of priority from DE 10 2024 109 128.2 filed on Mar. 29, 2024, the entire contents of which is incorporated herein by reference.

### BACKGROUND

### Field

The present disclosure relates to reprocessing medical instruments. More specifically, the present disclosure relates to reprocessing machines, reprocessing trays, and fixation elements for holding a medical instrument during reprocessing.

### Prior Art

Reusable medical instruments need to be carefully reprocessed after each use, before they can used on another patient. Reprocessing usually involves several steps, including manual precleaning and automated disinfection or sterilization. The automated disinfection or sterilization is usually performed in sophisticated reprocessing machines or autoclaves, where the medical instruments are treated with heated fluids, like liquids, gases, steam, plasma, and/or chemicals.

During automated disinfection of sterilization, medical instruments undergoing reprocessing are usually held in reprocessing trays, which are also used for transporting the medical instruments to and from the reprocessing machine or autoclave.

In order to enable effective reprocessing, the medical instruments undergoing reprocessing must be held in a suitable position during treatment. Therefore, reprocessing trays are equipped with fixation elements, which hold medical instruments in the desired position. The desired position may be a position which avoids direct contact between multiple medical instruments in the reprocessing tray, or between different sections of a deformable medical instrument, like a flexible endoscope.

For securely holding a medical instrument during transport and reprocessing, and at the same time providing for efficient reprocessing, it is essential that the medical instrument is placed on or in a reprocessing tray in the correct position. For more complex medical instruments, like flexible endoscopes, bringing the medical instrument into the correct position does not only require fixing different parts of the medical instrument with the correct fixation elements, but also requires doing so in a predefined sequence.

Especially with inexperienced operators, this may result in prolonged time required for placing medical instruments in reprocessing trays, and comes with a risk of errors.

It is therefore desirable to provide reprocessing machines, reprocessing trays, and fixation elements that address some of the aforementioned problems.

### SUMMARY

The present disclosure provides a reprocessing tray for holding medical instruments, comprising a tray body and a plurality of fixation elements, wherein the plurality of fixation elements are equipped with visual indicia, the visual indicia indicating one or more of the following: a sequence, in which the medical instrument is to be brought into contact with the respective fixation elements, and a portion of the medical instrument, which is to be brought into contact with the respective fixation elements.

With the visual indicia provided with the fixation elements described herein, a user can more easily recognize the correct position and/or sequence in which a medical instrument is to be placed in a reprocessing tray. The time required for correct placement can therefore be reduced and, at the same time, the risk of incorrect placement can be also reduced.

The visual indicia may include the fixation elements at least partially having different colors. In an embodiment, fixation elements to which the medical instrument needs to be fixed first may have a first color, like e.g., red, fixation elements to which the medical instrument needs to be fixed next may have a second color, like e g., yellow, and fixation elements to which the medical instrument needs to be fixed last may have a third color, like e.g., blue.

The visual indicia may include textual or numerical information being printed on, embossed in, engraved in, or otherwise associated with respective fixation elements. In an embodiment, a fixation element to which the medical instrument needs to be fixed first may be marked with a “1”, a fixation element to which the medical instrument needs to be fixed next may be marked with a “2”, and so on.

The visual indicia may include light emitting devices. The light emitting devices may comprise incandescent lamps, light emitting diodes (LED), laser diodes (LD), or any other suitable light source.

The fixation elements may further comprise sensors for detecting a part of the medical instrument being brought into contact with a respective fixation element. Sensors may be one or more of tactile sensors, like microswitches, inductive sensors, capacitive sensors, optical sensors, or any other suitable sensors.

The fixation elements may further comprise an energy storage. The energy storage may provide energy for the light emitting devices, so that the light emitting devices may be turned on or off. The energy storage may be a single-use energy storage. The single-use energy storage may be a primary battery. The single-use energy storage may be configured to provide enough energy so that the light emitting device can be operated a predetermined number of times.

The energy storage may be a rechargeable energy storage. The rechargeable energy storage may be a secondary battery. The rechargeable energy storage may be recharged a number of times when the rechargeable energy storage is depleted.

The reprocessing tray may further comprise a wireless charging receiver. The wireless charging receiver may receive energy from an electromagnetic field, and forward such energy to rechargeable energy storages for recharging. In some embodiments, the wireless charging receiver may be associated with the tray body, and coupled with the plurality of fixation elements. In other embodiments, a separate wireless charging receiver may be associated with each of the plurality of fixation elements.

The reprocessing tray may further comprise at least one controller for controlling activation states of the light emitting devices. The controller may be configured to switch the light emitting devices between two or more of: an “off” state, in which a light emitting device is turned off, a first “on” state, in which the light emitting device indicates that the medical device has been correctly been brought into contact with a respective fixation element, a second “on” state, in which the light emitting device indicates that the medical device has been incorrectly been brought into contact with a respective fixation element, and a third “on” state, in which the light emitting device indicates that the respective fixation element is the next to be brought into contact with the medical device. The first “on” state may be a state in which a light emitting device emits green light. The second “on” state may be a state in which a light emitting device emits yellow light. The third “on” state may be a state in which a light emitting element emits red light. With the respective activation states, a user may easily be guided through the correct procedure of placing a medical instrument in a reprocessing tray.

The one or more sensors may be configured to detect one or more markers of a medical instrument, so that the one or more sensors can determine whether a correct portion of the medical instrument has been brought into contact with the respective fixation element. The markers may be optical markers, inductive markers, capacitive markers, RFID markers, or any other suitable marker.

At least one of the fixation elements may comprise a body made from an elastic polymer. The elastic polymer can comprise silicone. Fixation elements comprising elastic polymers may elastically engage parts of a medical instrument without damaging the surface thereof.

The polymer may be at least partially translucent. The translucent polymer may allow embedding a light source in the fixation element, so that the light source is visible when activated, while being protected from reprocessing media during reprocessing of the medical instrument.

An optical filler may be dispersed in the polymer. The optical filler may comprise light scattering particles. By dispersing an optical filler in the polymer of a fixation element, the activation state of a light emitting device embedded in the polymer may be more easily seen from different viewing angles.

The present disclosure also provides a fixation element of a reprocessing tray according to the above description.

The present disclosure further provides a reprocessing machine for medical instruments, comprising a reprocessing chamber for receiving a reprocessing tray holding one or more medical instruments, and a wireless charging transmitter provided in the reprocessing chamber. Providing a reprocessing machine with a wireless charging transmitter may allow for wireless charging of energy storages as described before. Providing a reprocessing machine with a wireless charging transmitter may also allow for charging medical instruments having rechargeable energy storages during reprocessing thereof.

### BRIEF DESCRIPTION OF THE DRAWINGS

The embodiments are now further explained with regard to exemplary drawings. The embodiments and drawings are only provided to facilitate better understanding of the concepts described herein, and are not intended to limit the scope of the appending claims. In the figures:

FIG. 1 illustrates a system and method for reprocessing a medical instrument;

FIG. 2 illustrates a medical instrument placed in a reprocessing tray;

FIGS. 3a to 3c illustrate fixation elements in an enlarged view,

FIG. 4 illustrates a further fixation element in a sectional view, and

FIG. 5 illustrates a further reprocessing tray in a sectional view.

### DETAILED DESCRIPTION

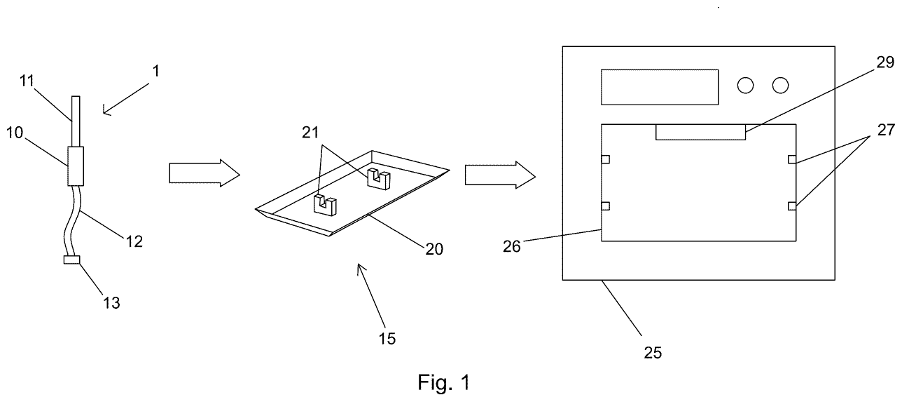

FIG. 1 shows a schematic overview of a reprocessing process for a medical instrument 1. In the shown example, the medical instrument 1 is a surgical video endoscope having a main body 10, an elongate shaft 11, a cable 12, and a plug 13.

After the medical instrument 1 has been used with a patient in a surgical procedure, it needs to be carefully reprocessed before reuse on a further patient, so that cross-contamination with pathogens can securely be prevented.

Reprocessing usually involves manual precleaning, wherein body fluids and other residues are removed from the surface and any internal channels of the medical instrument. Precleaning can be done using brushes, wipes, and low temperature detergents. The precleaning step is not shown in FIG. 1 as it is not essential for the understanding of the present disclosure.

After precleaning, the medical instrument 1 is placed in a reprocessing tray 15. The reprocessing tray 15 comprises a tray body 20 and a number of fixation elements 21. The tray body 20 may comprise a thermoplastic or thermosetting polymer, a sheet metal, a wire mesh, or a plastic mesh. In case of a polymer or sheet metal tray body, openings (not shown) may be provided in the tray body to enable diffusion of steam during reprocessing in an autoclave. Wire or plastic mesh trays are more commonly used for reprocessing in endoscope reprocessing machines using liquid reprocessing agents.

The fixation elements 21 are configured to secure the medical instrument 1 in the reprocessing tray 15. Usually, the fixation elements 21 comprise bodies of an elastic polymer like silicone, which is sufficiently soft to prevent any scratching or cutting of the surface of the medical instrument 1. The fixation elements 21 must hold the medical instrument 1 strong enough to prevent dislodging of the medical instrument 1.

The reprocessing tray 15 with the medical instrument 1 placed therein is then transported to an automatic reprocessing device 25, like an endoscope reprocessing machine or an autoclave.

The reprocessing device 25 has an internal reprocessing chamber 26, into which the reprocessing tray 15 can be inserted through a door (not shown). The reprocessing chamber 26 may be equipped with rails 27 for holding one or more the reprocessing trays 15. A wireless charging transmitter 29, which is described later in more detail, may be provided in the reprocessing chamber 26.

In the processing chamber, the medical instrument 1 is subjected to treatment with chemical detergents, disinfectants, water, gas, plasma, and/or steam at elevated temperatures. For effective reprocessing, the substances used for reprocessing must be able to reach all places of a surface of the medical instrument 1. However, the fixation elements 21 may block access to the surface in places where the fixation elements 21 touch the medical instrument 1. Further, flexible elements of the medical instrument 1, like the cable 12, may also block access to other surface sections if incorrectly placed. It is therefore of great importance that the medical instrument 1 is placed in the reprocessing tray 20 in the correct position, and for more complex instruments, also in the correct sequence.

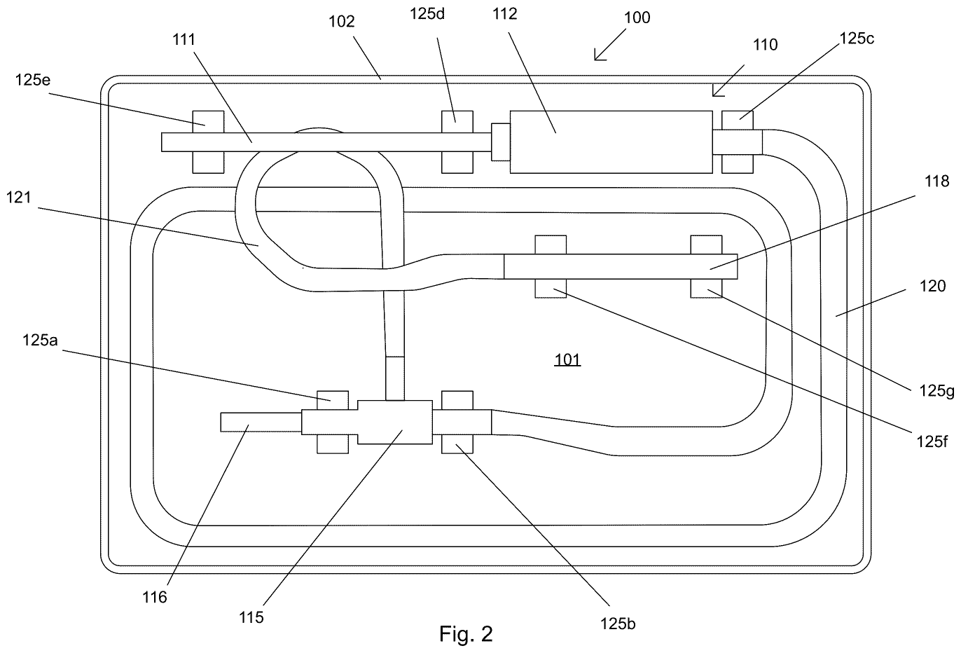

FIG. 2 shows a further reprocessing tray 100 comprising a base plate 101 and a wall 102 surrounding the base plate 101.

A video laparoscope 110 is placed in the reprocessing tray 100. The video laparoscope 110 comprises an elongated shaft 111, a handle 112, a connection body 115 with a light source plug 116, and a camera controller (CCU) plug 118. A first cable 120 connects the handle 112 and the connection body 115, and a second cable 121 connects the connection body 115 and the CCU plug 118.

On the base plate 101 of the reprocessing tray 100, fixation elements 125a to 125g are provided for securely holding the video laparoscope 110.

The video laparoscope 110 must be placed in the reprocessing tray 100 in a predetermined sequence, so that effective reprocessing can be warranted. First, the connection body 115 needs to be placed in the fixation elements 125a, 125b. Then, the cable 120 needs to be laid in a double loop and the handle 112 and the shaft 111 must be placed in fixation elements 125c, 125d, and 125e. Finally, the cable 121 is laid in a single loop and the CCU plug 118 is placed in the fixation elements 125f, 125g. While not shown in FIG. 2, fixation elements 125c, 125d, and 125e hold the handle 112 and the shaft 111 in an elevated position, so that the loop of cable 121 neatly slips underneath the shaft 111.

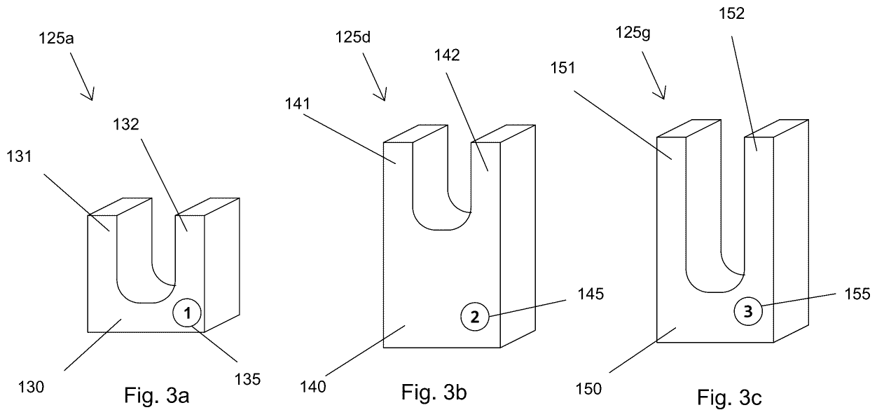

FIGS. 3a to 3c show fixation elements in an enlarged perspective view. FIG. 3a shows the fixation element 125a. The fixation element 125a comprises a base 130 and clamping arms 131, 132 extending from the base 130. The base 130 is configured to be fixed on the base plate 101 of the reprocessing tray 100 by suitable means (not shown) like one or more screws, magnets, pins, or the like. The base 130 and the clamping arms 131, 132 comprise, or are coated with, an elastic polymer like silicone. A visual indicia 135 is printed on, embossed or engraved in, or otherwise applied to a surface of the fixation element 125a. In the shown embodiment, the indicia 135 comprises the digit “1”, indicating that the fixation element 125a is in a first group of fixation elements in which a part of the video laparoscope 110 is to be placed.

FIG. 3b shows the fixation element 125d. Similar to fixation element 125a, the fixation element 125d comprises a base 140 and clamping arms 141, 142 extending therefrom. The base 140 of fixation element 125d is higher than the base of fixation element 125a, so that the shaft 111 of the video laparoscope 110 is held in an elevated position compared to the connection body 115. A visual indicia 145 is printed on, embossed or engraved in, or otherwise applied to a surface of the fixation element 125d. In the shown embodiment, the indicia 145 comprises the digit “2”, indicating that the fixation element 125d is in a second group of fixation elements in which a part of the video laparoscope 110 is to be placed.

FIG. 3c shows the fixation element 125g. Similar to fixation elements 125a and 125d, the fixation element 125g comprises a base 150 and clamping arms 151, 152 extending therefrom. While the base 150 of fixation element 125g has a similar hight as the base of fixation element 125a, the clamping arms 151, 152 are much longer, to accommodate the plate-like shape of the CCU plug 118. A visual indicia 155 is again printed on, embossed or engraved in, or otherwise applied to a surface of the fixation element 125g. In the shown embodiment, the indicia 155 comprises the digit “3”, indicating that the fixation element 125g is in a third group of fixation elements in which a part of the video laparoscope 110 is to be placed.

In FIGS. 3a to 3c, the visual indicia 135, 145, 155 are shown on a front surface of the base 130, 140, 150 of the respective fixation elements 125a, 125d, 125g In other embodiments, the visual indicia 135, 145, 155 may be provided in other places, like at the upper end surfaces of clamping arms 131, 132, 141, 142, 151, 152.

Instead of, or in addition to digital visual indicia shown in FIGS. 3a to 3c, the fixation elements 125a, 125d, 125g may be provided with other visual indicia, which may include a color coding. In some examples, a first group of fixation elements may be provided in red color, a second group of fixation elements may be provided in yellow color, and a third group of fixation elements may be provided in blue color.

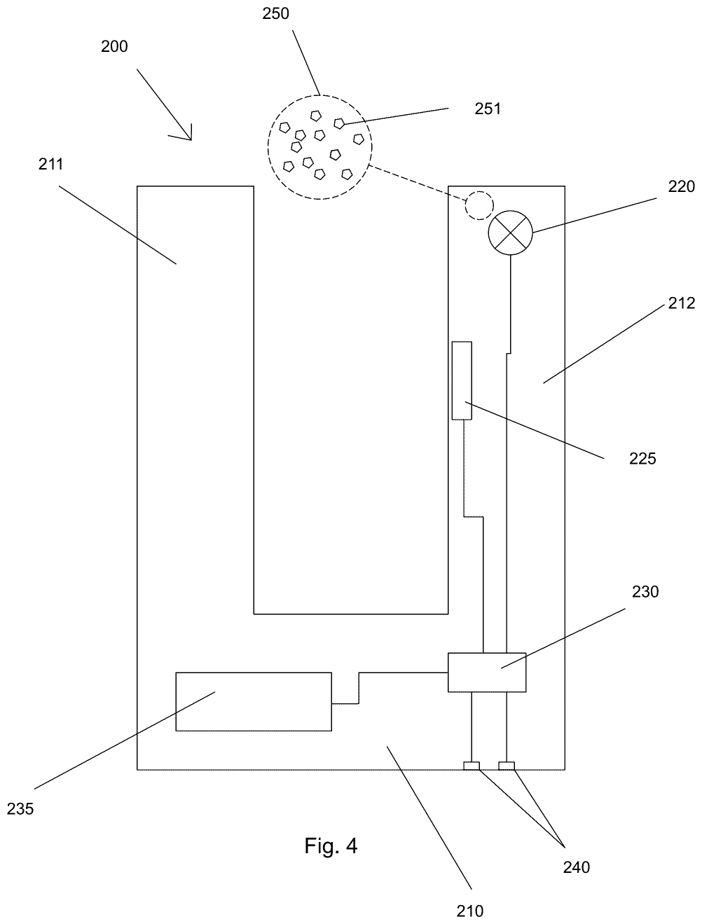

FIG. 4 shows a fixation element 200 according to a further embodiment. Again, the fixation element 200 comprises a base 210 and clamping arms 211, 212 made from a translucent elastic polymer like silicone.

Embedded in the fixation element 200, there is a light emitting device 220, a sensor 225, a microcontroller 230, and an energy storage 235.

The sensor 225 is configured to detect engagement of the fixation element 200 with a part of a medical instrument (not shown in FIG. 4). The sensor 225 may comprise a micro switch placed near an inner surface of the clamping arm 212, so that it can be activated by contact of the clamping arm 212 with the medical instrument. In other embodiments, the sensor 225 may be a contact-free sensor like a capacitive or inductive sensor, or a reed switch. In even further embodiments, the sensor 225 may be configured to identify labels (not shown) provided on the medial instrument, so that the sensor 225 may detect which part of the medical instrument is placed in the fixation element 20. The sensor 225 may comprise an RFID interrogator, a NFC interface, or any other suitable device.

The sensor 225 is connected to the microcontroller 230. The microcontroller 230 controls the light emitting device 220 depending on information received from the sensor 225.

The light emitting device 220 may be any suitable light source device. In some embodiments, the light emitting device 220 comprises one or more LEDs. The light emitting device 220 may comprise a red LED and a green LED, which can selectively be activated by the microcontroller 230 to emit green light, red light, or yellow light.

The energy storage 235 may be a primary battery, a secondary battery, or any other suitable energy storage. The energy storage 135 may comprise a small-scale nuclear battery, like a ⁶³Ni-based radionuclide battery as introduced by Betavolt Technology in 2024. The energy storage 235 may be a lithium-based secondary battery. The fixation element 200 may comprise contact pads 240 for receiving a charging current for recharging the battery storage 235 when it is depleted. The contact pads 240 may also be employed for communication between fixation device 200 and a main controller, as explained further below.

As shown in detail 250, an optical filler 251 may be dispersed in the polymer of the fixation element 200. The optical filler may comprise small particles for reflecting or scattering light emitted by the light emitting device 220. The optical filler 251 may improve visibility of light emitted by the light emitting device 220 from various different viewing angles.

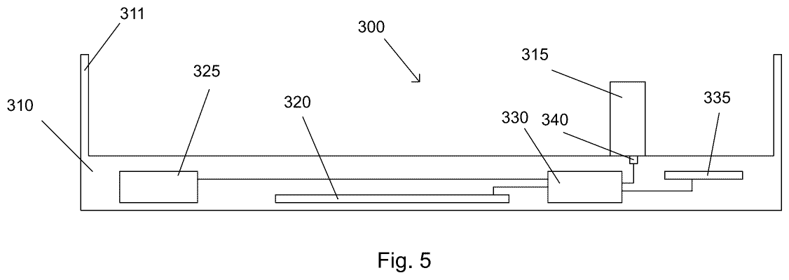

FIG. 5 shows a further reprocessing tray 300 in a sectional view. The reprocessing tray 300 comprises a base plate 310 and a wall 311. A fixation element 315 is shown to be mounted on the base plate 310, further fixation elements (not shown) may be provided.

In the base plate 310 of the reprocessing tray 300, a wireless charging receiver 320 is provided. The wireless charging receiver 320 is shown to be embedded in the base plate 310, but may equally be placed on an upper or lower surface of the base plate. Further, an energy storage 325, the main controller 330, and an RFID interrogator 335 are provided. Like the wireless charging receiver 320, the energy storage 325, the main controller 330, and the RFID interrogator 335 may be embedded in the base plate 310, or placed on an upper or lower surface thereof. Conductive traces in or on the base plate 310 connect the wireless charging receiver 320, the energy storage 325, the controller 330, the RFID interrogator 335, and contact pads 340 for connecting with contact pads of the fixation element 315, similar to contact pads 240 shown in FIG. 4 with regard to fixation element 200.

When a medical instrument (not shown) like the video laparoscope 110 is to be placed in the reprocessing tray 300, the main controller 330 activates the RFID interrogator 335 to read out an RFID identification tag of the medical instrument. Such RFID identification tags are provided on medical instruments for traceability. Based on information read from the RFID identification tag, the main controller 330 determines the sequence in which the medical instrument is to be placed in the respective fixation elements. The sequence is then communicated to the microcontrollers of the fixation elements.

Microcontrollers of a first group of fixation elements to be loaded first may control the light emitting devices of the respective fixation elements to switch to a first activation state, indicating that the respective fixation elements should be loaded in a first step. The first activation state may be a state in which the light emitting devices emit yellow light.

When the sensor of a fixation element detects insertion of the medical instrument, the respective microcontroller may switch the light emitting device to a second activation state, in which the light emitting device may emit green light. The microcontroller may further communicate to the controller 300 that the fixation element has been correctly loaded.

When all fixation elements of the first group have reported correct loading, the controller 330 may communicate with microcontrollers of a second group of fixation elements to be loaded next. The respective microcontrollers can then switch their light emitting devices to the first activation state, until the associated sensors report correct loading. Such process is repeated until all fixation elements are correctly loaded.

If one of the sensors determines that a fixation element has been loaded in the wrong sequence, the corresponding microcontroller may switch the light emitting device to a third activation state, in which the light emitting device may emit red light.

In addition, or as an alternative, to the different color light emitting devices described above, different activation states of the light emitting devices may include periodic or aperiodic blinking of the light emitting devices under the control of the corresponding microcontroller. Different states of the respective fixation elements may be represented by different blinking frequencies, different pulse-pause-ratios, or the like.

Where the sensors are configured to detect labels of the medical instrument, a microcontroller may also switch the corresponding light emitting device to the third activation state when a wrong part of the medical instrument has been inserted into the corresponding fixation element.

After all fixation elements have been correctly loaded, the main controller may communicate with the microcontrollers of all fixation elements to switch the light emitting devices back into the “off” state, so that the energy storages are not unnecessarily depleted.

The correctly loaded reprocessing tray 300 can then be placed into the reprocessing chamber 26 of the reprocessing machine 25 for reprocessing of the medical instrument. During the reprocessing procedure, the wireless charging transmitter 29 of the reprocessing machine 25 may be activated to provide charging energy to the wireless charging receiver 320 of the reprocessing tray 300. The wireless charging receiver 320 can convert the charging energy into a charging current, which can then be distributed by the controller 330 to the energy storage 325 of the reprocessing tray 300 and/or to the energy storages 235 of the respective fixation elements.

In some embodiments, the controller 330 may be further configured to detect through suitable sensors (not shown) whether the reprocessing tray 300 is correctly loaded into the reprocessing chamber 26, and to communicate with the microcontrollers of the fixation elements to signal the correct or incorrect loading through the light emitting devices of some or all of the fixation elements. Again, such signalling can use different color of light emission, different frequencies, pulse-pause ratios, or the like. In a similar manner, other relevant status information related to the reprocessing tray 300, the reprocessing machine 25, the medical device 1, or the like, may be signalled through the light emitting devices of the fixation elements.

In some embodiments, charging energy may be provided to the reprocessing tray 300 outside of the reprocessing chamber, e.g. on a transport cart or in a separate charging station.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that can fall within the scope of the appended claims.

What is claimed is:

1. A reprocessing tray for holding a medical instrument, the reprocessing tray comprising:
a tray body; and
a plurality of fixation elements fixed to a surface of the tray body,
wherein each of the plurality of fixation elements comprise a visual indicia, the visual indicia indicating one or more of the following:
a sequence, in which the medical instrument is to be brought into contact with each of the plurality of fixation elements, and
a portion of the medical instrument, which is to be brought into contact with each of the plurality of fixation elements.

2. The reprocessing tray of claim 1, wherein the visual indicia comprises the plurality of fixation elements at least partially having different colors.

3. The reprocessing tray of claim 1, wherein the visual indicia comprises one of textual or numerical information disposed on each of the plurality of fixation elements.

4. The reprocessing tray of claim 1, wherein the visual indicia comprises a light emitting device.

5. The reprocessing tray of claim 4, wherein each of the plurality of fixation elements further comprise a sensor for detecting a part of the medical instrument.

6. The reprocessing tray of claim 4, wherein each of the plurality of fixation elements further comprise an energy storage for providing power to the light emitting device.

7. The reprocessing tray of claim 5, wherein each of the plurality of fixation elements further comprise an energy storage for providing power to the sensor.

8. The reprocessing tray of claim 6, wherein the energy storage comprises one of a single-use energy storage or a rechargeable energy storage.

9. The reprocessing tray of claim 7, wherein the energy storage comprises one of a single-use energy storage or a rechargeable energy storage.

10. The reprocessing tray of claim 8, further comprising a wireless charging receiver.

11. The reprocessing tray of claim 10, wherein the wireless charging receiver is associated with the tray body, and coupled with the plurality of fixation elements.

12. The reprocessing tray of claim 10, wherein a separate wireless charging receiver is associated with each of the plurality of fixation elements.

13. The reprocessing tray of claim 9, further comprising a wireless charging receiver.

14. The reprocessing tray of claim 13, wherein the wireless charging receiver is associated with the tray body, and coupled with the plurality of fixation elements.

15. The reprocessing tray of claim 13, wherein a separate wireless charging receiver is associated with each of the plurality of fixation elements.

16. The reprocessing tray of claim 4, further comprising at least one controller for controlling an activation state of the light emitting device.

17. The reprocessing tray of claim 16, wherein the controller is configured to switch the light emitting device between two or more of:
an off state, in which the light emitting device is turned off,
a first on state, in which the light emitting device indicates that the medical device has correctly been brought into contact with a respective fixation element,
a second on state, in which the light emitting device indicates that the medical device has incorrectly been brought into contact with a respective fixation element, and
a third on state, in which the light emitting device indicates that the respective fixation element is the next to be brought into contact with the medical device.

18. The reprocessing tray of claim 5, wherein the sensor is configured to detect one or more markers of a medical instrument, so that the sensor can determine whether a correct portion of the medical instrument has been brought into contact with the respective fixation element of the plurality of fixation elements.

19. The reprocessing tray of claim 1, wherein at least one of the plurality of fixation elements comprises a body made from an elastic polymer.

20. The reprocessing tray of claim 19, wherein the elastic polymer comprises silicone.

21. The reprocessing tray of claim 19, wherein the elastic polymer is at least partially translucent.

22. The reprocessing tray of claim 19, wherein an optical filler is dispersed in the elastic polymer.

23. A fixation element for use with a reprocessing tray, the fixation element comprising;
a visual indicia, the visual indicia indicating one or more of the following:
a sequence, in which the medical instrument is to be brought into contact with each of the plurality of fixation elements, and
a portion of the medical instrument, which is to be brought into contact with each of the plurality of fixation elements.

24. A reprocessing machine for medical instruments, the reprocessing machine comprising:
a reprocessing chamber for receiving a reprocessing tray holding one or more medical instruments, and
a wireless charging transmitter provided in the reprocessing chamber for charging an energy storage provided in a reprocessing tray accommodated with the reprocessing chamber.
